(19) United States
(12) Patent Application Publication
(10) Pub. No.: US 2009/0216043 A1
(43) Pub. Date: Aug. 27, 2009

(54) Arylglycerine Ether

(75) Inventors: Joachim Hess, Hofheim (DE); Ralf Zerrer, Karlstein (DE); Franz-Xaver Scherl, Burgkirchen (DE); Christian Sowa, Neustadt/ Weinstrasse (DE)

Correspondence Address: CLARIANT CORPORATION;INTELLECTUAL PROPERTY DEPARTMENT, 4000 MONROE ROAD, CHARLOTTE, NC, 28205, US

(73) Assignee: CLARIANT PRODUKTE (DEUTSCHLAND) GMBH, Frankfurt am Main (DE)

(21) Appl. No.: 11/922,855

(22) PCT Filed: Jun. 22, 2006
(86) PCT No.: PCT/EP2006/006003

§ 371 Date: Feb. 2, 2009

(30) Foreign Application Priority Data
Jun. 30, 2005 (DE) 10 2005 030 526.1

(51) Int. Cl. C07C 43/115; C07C 309/12

(52) U.S. Cl. 562/36; 568/660

(57) ABSTRACT

The invention relates to aromatically substituted glycerine ethers of formula (I),

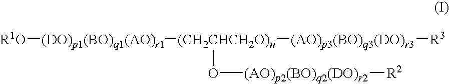

wherein at least one of the radicals R¹, R², or R³ is or contains an aromatic group. Said compounds can be used, preferably, as surface-active agents.

The object of the invention is aromatically substituted glycerine ethers and the use thereof in dispersions.

Dispersions are substance systems, consisting of two or several phases, wherein one substance (the dispersed or disperse phase) is distributed, i.e. dispersed, in very fine form in another (the dispersing agent or dispersant). Both dispersing agent and the substances of the disperse phase can be solid, liquid or gaseous. Depending on the degree of distribution, they are differentiated into molecular disperse systems, colloid disperse systems, fine disperse systems and coarse disperse systems. Examples of dispersions are suspensions, emulsions, foams, aerosols and smoke. Many industrial products are used in the form of dispersions.

In order to obtain stable dispersions, surface-active substances (dispersants) must often be added, which render possible at all, or facilitate, the distribution of the phases and counteract gel formation, phase separation, crystallization or sedimentation of the individual components.

Thus there is great interest in efficient dispersants which are readily available industrially and usable for different substance systems, but are also toxicologically safe, and harmless to the environment.

Surprisingly it has been found that aromatically substituted glycerine ethers display outstanding surfactant properties, are readily usable in aqueous dispersions, and also in emulsions, suspension concentrates and suspoemulsions, and result in high mutual compatibility of the components, for example adjuvants, dispersants, electrolytes, etc., and high suspendibility (ease of suspension) of the components, and stabilization of the phases.

The object of the invention is aromatically substituted glycerine ethers of the formula (I)

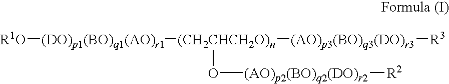

wherein

R¹, R² and R³ mutually independently can be the same or different and stand for hydrogen —H,

and/or for

—(CR⁴R⁵)ₓ phenyl, wherein R⁴ and R⁵, which can be the same or different, stand for —H, for an OH group, for linear or branched (C₁—Cl₀) alkyl or for linear or branched (C₂-C₃₀) alkenyl and x stands for a number from 1 to 10,

and/or for

—(CR⁴R⁵)ₓ naphthyl, wherein R⁴ and R⁵, which can be the same or different, stand for —H, for an OH group, for linear or branched (C₁-C₁₀) alkyl or for linear or branched (C₂-C₃₀) alkenyl and x stands for a number from 1 to 10,

and/or for

a group of the formula (II)

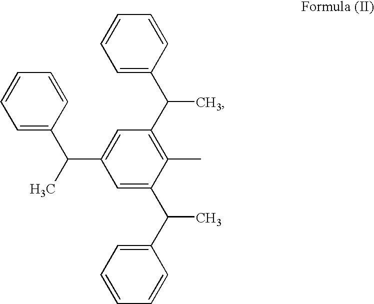

and/or for

R⁶R⁷N—(CH₂)_{y}—, wherein R⁶ and R⁷, which can be the same or different, stand for —H, linear or branched (C₁-C₁₀) alkyl or linear or branched (C₂-C₃₀) alkenyl and y stands for a number from 1 to 22,

and/or for

HO—(CH₂)_{y}—, wherein y stands for a number from 1 to 22,

and/or for

SO₃⁻X⁺, —PO₃²⁻X⁺X′⁺ or —CH₂COO⁻X⁺, wherein X⁺ and X′⁺ stand for H⁺, Li⁺, Na⁺, K⁺, Ca²⁺/2, Mg²⁺/2 or N(R⁸)₄⁺ and the R⁸ can be the same or different and stand for —H or (C₁-C₁₀) alkyl, preferably (C₁-C₄) alkyl,

and/or for

a group of the formula (III)

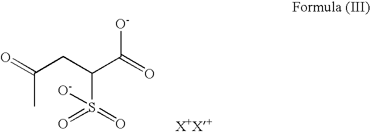

wherein X⁺ and X′⁺ stand for H⁺, Li⁺, Na⁺, K⁺, Ca²⁺/2, Mg²⁺/2 or N(R⁸)₄⁺ and the R⁸ can be the same or different and stand for —H or (C₁-C₁₀) alkyl, preferably (C₁-C₄) alkyl,

and/or for

—C(R⁹)₂—COO⁻X⁺, —CO—R¹⁰—COO⁻X⁺ or —C(R⁹)₂C(R⁹)₂C(R⁹)₂—N(R¹¹)₂, wherein the R⁹ can be the same or different and stand for —H and/or —CH₃, R¹⁰ stands for (C₁-C₁₀) alkylene or (C₂-C₃₀) alkenylene, the R¹¹ can be the same or different and stand for (C₁-C₁₀) alkyl or (C₂-C₃₀) alkenyl, X⁺ stands for H⁺, Li⁺, Na⁺, K⁺, Ca²⁺/2, Mg²⁺/2 or N(R⁸)₄⁺ and the R⁸ can be the same or different and stand for —H or (C₁-C₁₀) alkyl, preferably for (C₁-C₄) alkyl,

and/or for

—C(R¹²)₂C(R¹²)₂C(R¹²)₂—N((GO)_{z}H)₂, wherein the R¹² can be the same or different and stand for —H and/or —CH₃, G stands for —C₂H₄—, —C₃H₆— or —C₄H₈— and z for a number from 1 to 22,

and/or stand for

a group of the formula (IV)

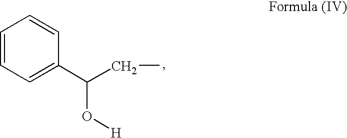

A, B and D, which can be the same or different, stand for —C₂H₄—, —C₃H₆—, —C₄H₈— or —CH(phenyl)CH₂—,

the indices p1, q1, r1, p2, q2, r2, p3, q3 and r3 stand for numbers from 0 to 500, and n stands for a number from 1 to 100,

with the proviso that at least one of the residues R¹, R² and R³ is or contains an aromatic group.

In a preferred embodiment of the invention, the compounds of the formula (I) contain one or more of the residues R¹, R² and R³ selected from benzyl, the group of the formula (II), the group of the formula (III), —PO3²⁻X⁺X′⁺, —SO₃⁻X⁺ and the group of the formula (IV). In a particularly preferred embodiment of the invention, the compounds of the formula (I) contain one or several of the residues R¹, R² and R³ selected from benzyl, the group of the formula (II) and the group of the formula (IV).

Preferably n stands for a number from 1 to 35 and particularly preferably for a number from 2 to 35. In a particularly preferred embodiment of the invention n stands for a number from 2 to 5. In a further particularly preferred embodiment of the invention, n stands for a number from 4 to 6. In a further particularly preferred embodiment of the invention, n stands for a number from 5 to 25. Within the last-named range, the range from 5 to 10 is exceptionally preferred.

In a further preferred embodiment of the invention, A, B and D, which can be the same or different, each mutually independently stands for —C₂H₄— or —C₃H₆— and preferably for —C₂H₄—.

In a further preferred embodiment of the invention, the indices p1, q1, r1, p2, q2, r2, p3, q3 and r3 stand for numbers from 0 to 100, preferably from 0 to 50, particularly preferably from 0 to 25 and especially preferably from 0 to 20.

In a further preferred embodiment of the invention the sum of the indices p1, q1, r1, p2, q2, r2, p3, q3 and r3 is a number from 0 to 100, preferably from 0 to 60 and particularly preferably from 0 to 40.

In a particularly preferred embodiment of the invention, the aromatically substituted glycerine ethers of the formula (I) are compounds wherein the residues R¹, R² and R³ are selected from —H and benzyl —CH₂-phenyl and one or more, preferably two, of the residues R¹, R² and R³ stand for benzyl,

A, B and D stand for —CH₂CH₂—,

the indices p1, q1, r1, p2, q2, r2, p3, q3 and r3 stand for numbers from 0 to 25, preferably 0 to 20, and the sum of the indices p1, q1, r1, p2, q2, r2, p3, q3 and r3 is a number from 0 to 30, preferably from 5 to 30 and particularly preferably 15 to 20,

n stands for a number from 2 to 5,

and wherein the benzyl groups are directly bound to the glycerine unit and in the case where the residue R² stands for benzyl, one or more, of the residues R² stands for benzyl.

Especially preferred are aromatically substituted glycerine ethers of the formula (I), wherein R¹ and R² stand for benzyl, R³ stands for —H, n stands for a number from 2 to 5, p1, q1, r1, p2, q2, r2 stand for the number 0, A, B and D stand for —CH₂CH₂—, p3, q3 and r3 stand for numbers from 0 to 25, preferably 0 to 20, and the sum of the indices p3, q3 and r3 is a number from 0 to 30, preferably 5 to 30 and particularly preferably 15 to 20.

In a further particularly preferred embodiment of the invention, the aromatically substituted glycerine ethers of the formula (I) are compounds wherein the residues R¹, R² and R³ are selected from —H and the group of the formula (II) and one or more, preferably one, of the residues R¹, R² and R³ stands for the group of the formula (II),

A, B and D, which can be the same or different, stand for —C₂H₄—, —C₃H₆—, —C₄H₈— or —CH(phenyl)CH₂—, preferably for —C₂H₄—,

the indices p1, q1, r1, p2, q2, r2, p3, q3 and r3 stand for numbers from 0 to 100, preferably from 0 to 50 and particularly preferably from 0 to 25, and the sum of the indices p1, q1, r1, p2, q2, r2, p3, q3 and r3 is a number from 0 to 100, preferably from 10 to 60, particularly preferably from 15 to 40,

n stands for a number from 1 to 8, preferably for 2 to 5,

and wherein in the case where the residue R stands for the group of the formula (II), one or more of the residues R² stand for the group of the formula (II).

Further especially preferred are aromatically substituted glycerine ethers of the formula (I), wherein R¹ stands for a group of the formula (II), R² and R³ stand for —H, A, B and D, which can be the same or different, stand for —C₂H₄—, —C₃H₆—, —C₄H₈— or —CH(phenyl)CH₂—, preferably for —C₂H₄—, the indices p1, q1, r1, p2, q2, r2, p3, q3 and r3 stand for numbers from 0 to 100, preferably from 0 to 50 and particularly preferably from 0 to 25, the sum of the indices p1, q1, r1, p2, q2, r2, p3, q3 and r3 is a number from 0 to 100, preferably from 10 to 60, particularly preferably from 15 to 40, and n stands for a number from 1 to 8, preferably for 2 to 5.

In a further particularly preferred embodiment of the invention, the aromatically substituted glycerine ethers of the formula (I) are compounds wherein one or more, preferably one, of the residues R¹, R² or R³ stands for a group of the formula (III),

A, B and D, which can be the same or different, stand for —C₂H₄—, —C₃H₆—, —C₄H₈— or —CH(phenyl)CH₂—, preferably for —C₂H₄—,

the indices p1, q1, r1, p2, q2, r2, p3, q3 and r3 stand for numbers from 0 to 100, preferably from 0 to 50 and particularly preferably from 0 to 25, and the sum of the indices p1, q1, r1, p2, q2, r2, p3, q3 and r3 is a number from 0 to 100, preferably from 10 to 60, particularly preferably from 15 to 40,

n stands for a number from 2 to 10, preferably for a number from 4 to 9 and particularly preferably for a number from 4 to 6,

and wherein in the case where the residue R² has a meaning other than —H, just one or else also more of the residues R² can have a meaning other than —H.

Further especially preferred are aromatically substituted glycerine ethers of the formula (I), wherein R¹ stands for a group of the formula (II), one of the residues R² or R³ stands for a group of the formula (III) and all remaining residues R² or R³ stand for —H, A, B and D, which can be the same or different, stand for —C₂H₄—, —C₃H₆—, —C₄H₈— or —CH(phenyl)CH₂—, preferably for —C₂H₄—, the indices p1, q1, r1, p2, q2, r2, p3, q3 and r3 stand for numbers from 0 to 100, preferably from 0 to 50 and particularly preferably from 0 to 25, and the sum of the indices p1, q1, r1, p2, q2, r2, p3, q3 and r3 is a number from 0 to 100, preferably from 10 to 60, particularly preferably from 15 to 40, and n stands for a number from 2 to 10, preferably for a number from 4 to 9 and particularly preferably for a number from 4 to 6.

In a further particularly preferred embodiment of the invention, the aromatically substituted glycerine ethers of the formula (I) are compounds wherein one or more, preferably one, of the residues R¹, R² and R³ stands for —PO₃²⁻X⁺X′⁺, wherein X⁺ and X′⁺ stand for H⁺, Li⁺, Na⁺, K⁺, Ca²⁺/2, Mg²⁺/2 or N(R⁸)₄⁺ and the R⁸ can be the same or different and stand for —H or (C₁-C₁₀) alkyl, preferably (C₁-C₄) alkyl,

A, B and D, which can be the same or different, stand for —C₂H₄—, —C₃H₆—, —C₄H₈— or —CH(phenyl)CH₂—, preferably for —C₂H₄—,

the indices p1, q1, r1, p2, q2, r2, p3, q3 and r3 stand for numbers from 0 to 100, preferably from 0 to 50 and particularly preferably from 0 to 25, and the sum of the indices p1, q1, r1, p2, q2, r2, p3, q3 and r3 is a number from 0 to 100, preferably from 10 to 60, particularly preferably from 15 to 40,

n stands for a number from 2 to 10, preferably for a number from 4 to 9 and particularly preferably for a number from 4 to 6,

and wherein in the case where the residue R² has a meaning other than —H, just one or else also more of the residues R² can have a meaning other than —H.

Further especially preferred are aromatically substituted glycerine ethers of the formula (I), wherein R¹ stands for a group of the formula (II), one of the residues R² or R³ stands for —PO₃²⁻X⁺X′⁺, wherein X⁺ and X′⁺ stand for H⁺, Li⁺, Na⁺, K⁺, Ca²⁺/2, Mg²⁺/2 or N(R⁸)₄₊ and the R⁸ can be the same or different and stand for —H or (C₁-C₁₀) alkyl, preferably (C₁-C₄) alkyl, and all remaining residues R² or R³ stand for —H, A, B and D, which can be the same or different, stand for —C₂H₄—, —C₃H₆—, —C₄H₈— or

—CH(phenyl)CH₂—, preferably for —C₂H₄—, the indices p1, q1, r1, p2, q2, r2, p3, q3 and r3 stand for numbers from 0 to 100, preferably from 0 to 50 and particularly preferably from 0 to 25, and the sum of the indices p1, q1, r1, p2, q2, r2, p3, q3 and r3 is a number from 0 to 100, preferably from 10 to 60, particularly preferably from 15 to 40, and n stands for a number from 2 to 10, preferably for a number from 4 to 9 and particularly preferably for a number from 4 to 6.

In a further particularly preferred embodiment of the invention, the aromatically substituted glycerine ethers of the formula (I) are compounds wherein one or more, preferably one, of the residues R¹, R² and R³ is —SO₃⁻X⁺, wherein X⁺ stands for H⁺, Li⁺, Na⁺, K⁺, Ca²⁺/2, Mg²⁺/2 or N(R⁸)₄₊ and the R⁸ can be the same or different and stand for —H or (C₁-C₁₀) alkyl, preferably (C₁-C₄) alkyl, A, B and D, which can be the same or different, stand for —C₂H₄—, —C₃H₆—, —C₄H₈— or —CH(phenyl)CH₂—, preferably for —C₂H₄—,

the indices p1, q1, r1, p2, q2, r2, p3, q3 and r3 stand for numbers from 0 to 100, preferably from 0 to 50 and particularly preferably from 0 to 25, and the sum of the indices p1, q1, r1, p2, q2, r2, p3, q3 and r3 is a number from 0 to 100, preferably from 10 to 60, particularly preferably from 15 to 40,

n stands for a number from 2 to 10, preferably for a number from 4 to 9 and particularly preferably for a number from 4 to 6,

and wherein in the case where the residue R² has a meaning other than —H, just one or else also more of the residues R² can have a meaning other than —H.

Further especially preferred are aromatically substituted glycerine ethers of the formula (I), wherein one or more, preferably one, of the residues R¹, R² and R³ stands for a group of the formula (II) and one or more, preferably one, of the residues R¹, R² and R³ for —SO₃⁻X⁺, wherein X⁺ stands for H⁺, Li⁺, Na⁺, K⁺, Ca²⁺/2, Mg²⁺/2 or N(R⁸)₄⁺ and the R⁸ can be the same or different and stand for —H or (C₁-C₁₀) alkyl, preferably (C₁-C₄) alkyl, and all remaining residues R¹, R² or R³ stand for —H, A, B and D, which can be the same or different, stand for —C₂H₄—, —C₃H₆—, —C₄H₈— or —CH(phenyl)CH₂—, preferably for —C₂H₄—, the indices p1, q1, r1, p2, q2, r2, p3, q3 and r3 stand for numbers from 0 to 100, preferably from 0 to 50 and particularly preferably from 0 to 25, and the sum of the indices p1, q1, r1, p2, q2, r2, p3, q3 and r3 is a number from 0 to 100, preferably from 10 to 60, particularly preferably from 15 to 40, and n stands for a number from 2 to 10, preferably for a number from 4 to 9 and particularly preferably for a number from 4 to 6.

In a further particularly preferred embodiment of the invention, the aromatically substituted glycerine ethers of the formula (I) are compounds wherein the residues R¹, R² and R³ are selected from —H and the group of the formula (IV) and one or more, preferably two, of the residues R¹, R² and R³ stand for a group of the formula (IV),

A, B and D stand for —CH₂CH₂—,

the indices p1, q1, r1, p2, q2, r2, p3, q3 and r3 stand for numbers from 0 to 25, preferably 0 to 20, and the sum of the indices p1, q1, r1, p2, q2, r2, p3, q3 and r3 is a number from 0 to 30, preferably 5 to 30 and particularly preferably 15 to 20,

n stands for a number from 1 to 5,

and wherein the groups of the formula (IV) are bound directly to the glycerine unit and in the case where the residue R² stands for the group of the formula (IV), one or more of the residues R² stand for the group of the formula (IV).

Further particularly preferred are aromatically substituted glycerine ethers of the formula (I), wherein R¹ and R² stand for a group of the formula (IV), R³ stands for —H, n stands for a number from 1 to 5, p1, q1, r1, p2, q2, r2 stand for the number 0, A, B and D stand for —CH₂CH₂—, p3, q3 and r3 stand for numbers from 0 to 25, preferably 0 to 20, and the sum of the indices p3, q3 and r3 is a number from 0 to 30, preferably 5 to 30 and particularly preferably 15 to 20.

In a further particularly preferred embodiment of the invention, the aromatically substituted glycerine ethers of the formula (I) are compounds wherein the residues R¹, R² and R³ are selected from —H and the group of the formula (IV) and one or more, preferably two, of the residues R¹, R² and R³ stand for a group of the formula (IV),

A, B and D are selected from —CH₂CH₂— and —CH(phenyl)CH₂— and wherein in the case where the glycerine ethers contain both —CH₂CH₂— and also —CH(phenyl)CH₂ groups, —CH₂CH₂— is bound directly to the glycerine unit and —CH(phenyl)CH₂— to ethyleneoxy,

the indices p1, q1, r1, p2, q2, r2, p3, q3 and r3 stand for numbers from 0 to 25, preferably 0 to 20, and the sum of the indices p1, q1, r1, p2, q2, r2, p3, q3 and r3 is a number from 0 to 35, preferably 5 to 35 and particularly preferably 15 to 25, and n stands for a number from 1 to 5,

and wherein the groups of the formula (IV) are bound directly to the glycerine unit and in the case where the residue R² stands for the group of the formula (IV), one or more of the residues R² stand for the group of the formula (IV).

Further especially preferred are aromatically substituted glycerine ethers of the formula (I), wherein R¹ and R² stand for a group of the formula (IV), R³ stands for —H, n stands for a number from 1 to 5, p1, q1 and r1 stand for the number 0, A stands for —CH₂CH₂—, B and D stand for —CH(phenyl)CH₂—, p2, q2, r2, p3, q3 and r3 stand for numbers from 0 to 25, preferably 0 to 20, and the sum of the indices p2, q2, r2, p3, q3 and r3 is a number from 0 to 35, preferably 5 to 35 and particularly preferably 15 to 25.

A further object of the present invention is dispersions containing one or more of the aromatically substituted glycerine ethers according to the invention.

The aromatically substituted glycerine ethers according to the invention are advantageously suitable as surfactant substances. A further object of the present invention is thus also the use of one or more of the aromatically substituted glycerine ethers according to the invention as surfactant substances.

In a further preferred embodiment of the invention the aromatically substituted glycerine ethers according to the invention are used as surfactant substances in dispersions.

### General Production Method

### A) Polymerization of Glycerine to Oligoglycerines or Polyglycerines

The polymerization of glycerine to oligoglycerines or polyglycerines is effected in a known manner.

For this, glycerine is heated in the presence of acidic catalysts, for example HCl, H₂SO₄, sulfonic acids or H₃PO₄ or in the presence of alkaline catalysts such as sodium hydroxide, potassium hydroxide, alkali metal alcoholates, alkali metal carbonates or alkali metal bicarbonates in a concentration range from 0.1 to 0.4 wt. % catalyst in a stirred apparatus with water separator and nitrogen flow at 200 to 280° C., preferably 240 to 270° C. With the removal of water condensation, the formation of the oligoglycerine or polyglycerine with a mean condensation level of 2 to 100, preferably 3 to 35 glycerine units, takes place within 3 to 14 hours. The mean molecular weight of the oligo- or polyglycerines can be calculated from the OH number.

The ratio of the condensation level n to the condensation time in the polymerization of glycerine to oligoglycerines or polyglycerines is shown in Table 1.

TABLE 1
Ratio of condensation level n to condensation time

| Condensation | OH number | Molar mass | Condensation time |
|---|---|---|---|
| level n | [mg KOH/g] | [g/mol] | [hours] |
| 2.0 | 1352 | 166 | 3-4 |
| 3.0 | 1169 | 240 | 3-4 |
| 4.0 | 1072 | 314 | 4-5 |
| 5.0 | 1012 | 388 | 5-7 |
| 6.0 | 971 | 462 | 6-7 |
| 7.0 | 942 | 536 | 7-9 |
| 8.0 | 920 | 610 | 8-9 |
| 9.0 | 902 | 684 | 9-11 |
| 10 | 888 | 758 | 10-11 |
| 11 | 877 | 832 | 11-12 |
| 12 | 867 | 906 | 11-12 |
| 13 | 859 | 980 | 12-13 |
| 14 | 850 | 1056 | 12-13 |
| 15 | 844 | 1130 | 13-14 |

### B) Production of Glycerine Ethers

For the production of the mono-, oligo- or polyglycerine ethers according to the invention, the aforesaid mono-, oligo- or polyglycerines or the corresponding alkoxylated glycerines can be etherified by an etherification method known to the skilled person, such as for example the Williamson ether synthesis. In the Williamson ether synthesis, the glycerines can for example be reacted with arylalkyl chlorides such as for example benzyl chloride under alkaline catalysis preferably at 80 to 110° C. The mono-, oligo- or polyglycerines can however for example also be reacted with appropriate oxides or epoxides under acidic or alkaline catalysis. In each case, the reaction is monitored by determination of the OH number.

The production of the mono-, oligo- or polyglycerine ethers according to the invention can also be effected by reacting the aforesaid mono-, oligo- or poly-glycerines or the corresponding alkoxylated glycerines by the Williamson ether synthesis for example with arylalkyl chlorides under alkaline catalysis preferably at 80 to 110° C. and then reacting the glycerine ethers thus obtained for example with oxides or epoxides under acidic or alkaline catalysis.

The mono-, oligo- or polyglycerine ethers according to the invention can be modified by standard methods known to the skilled person by alkoxylation, i.e. by introduction of alkyleneoxy groups, such as for example ethyleneoxy groups, by sulfation, phosphation, amination, etc.

The compounds according to the invention are characterized by excellent dispersant power and high electrolyte stability. In addition, the glycerine ethers according to the invention result in an improvement in the compatibility of hydrophilic and hydrophobic components and an increase in the wetting and absorption power of formulations which contain these glycerine ethers.

### EXAMPLES

Examples of the production of glycerine ethers according to the invention are described below, without restricting the invention thereto.

### Production of Oligo- or Polyglycerine Benzyl Ethers

### Production Procedure

The oligoglycerine to be reacted (oligoglycerine mixture with a mean condensation level n of 2 or 5) is treated with NaOH and stirred at 80° C. for 2 hours under water-pump vacuum. Next, benzyl chloride is added dropwise over 5 hours at normal pressure at a temperature of 80 to 100° C. During this, the temperature of 100° C. should not be exceeded. Next the mixture is stirred for a further 3 hours at 100° C. For the workup, the reaction mixture is shaken with water. The organic phase was isolated and dried.

The quantities of oligo- or polyglycerine PG, wherein n means the condensation level, of benzyl chloride and of sodium hydroxide NaOH used and the OH number of the glycerine ethers are shown in Table 2.

TABLE 2
Quantities of oligo- or polyglycerine, benzyl chloride and sodium
hydroxide used, and OH number of the glycerine ethers

| PG | | | benzyl chloride | | NaOH | | OH number |
|---|---|---|---|---|---|---|---|
| n | [mol] | [g] | [mol] | [g] | [mol] | [g] | [mg KOH/g] |
| 2 | 1.0 | 166.2 | 2.0 | 253.2 | 2.0 | 80.0 | 360.9 |
| 5 | 1.0 | 388.4 | 3.0 | 379.8 | 3.0 | 120.0 | 431.6 |

### Production of Oligo- or Polyglycerine Tristyrylphenyl Ethers

### General Production Procedure

The oligoglycerine to be reacted (oligoglycerine mixture with a mean condensation level n of 2 or 5) and tristyrylphenol or tristyrylphenol ethoxylate is treated with catalytic quantities of NaOH and heated with stirring at 170° C. Water of reaction formed is removed from the reaction mixture in the water separator. The reaction mixture is maintained at this temperature for a period of 6 hours. After this, it is cooled to room temperature.

The quantities of oligo- or polyglycerine PG, wherein n means the condensation level, of tristyrylphenol or tristyrylphenol ethoxylate TSP-(EO)ₘ—H, wherein EO means —CH₂CH₂O— used, and the OH number of the glycerine ethers are shown in Table 3.

TABLE 3
Quantities of oligo- or polyglycerine and tristyrylphenol or tristyrylphenol
ethoxylate used and OH number of the glycerine ethers

| PG | | | TSP-(EO)ₘ-H | | | OH number |
|---|---|---|---|---|---|---|
| n | [mol] | [g] | m | [mol] | [g] | [mg KOH/g] |
| 2 | 1.0 | 166.2 | 0 | 1.0 | 406.6 | 349.4 |
| 5 | 1.0 | 388.4 | 10 | 2.0 | 1694.2 | 312.1 |

### Production of Mono-, Oligo- or Polyglycerine Styrene Oxide Ethers

### General Production Procedure

Mono- or oligoglycerine (oligoglycerine mixture with a mean condensation level n of 5) is treated with potassium methoxide and heated at 70° C. Methanol which forms is drawn off under water-pump vacuum. After this, styrene oxide is added dropwise at 70 to 75° C. within a period of 3 to 5 hours. Next the mixture is stirred at 70 to 75° C. for a further 3 to 6 hours. Next the OH number is determined.

The quantities of mono-, oligo- or polyglycerine PG, wherein n means the condensation level, and of styrene oxide used, and the OH number of the glycerine ethers are shown in Table 4.

TABLE 4
Quantities of mono-, oligo- or polyglycerine and styrene
oxide used and OH number of the glycerine ethers

| | | |
|---|---|---|
| PG | Styrene oxide | OH number |

| n | [mol] | [g] | [mol] | [g] | [mg KOH/g] |
|---|---|---|---|---|---|
| 1 | 1.0 | 92.0 | 2.17 | 261 | 493.8 |
| 5 | 1.0 | 388.42 | 3.0 | 360.5 | 603.1 |

1. An aromatically substituted glycerine ether of the formula (I) 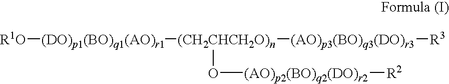
wherein
R¹, R² and R³ are the same or different and are
hydrogen —H,
—(CR⁴R⁵)ₓ phenyl, wherein R⁴ and R⁵ are the same or different, and are —H, an OH group, linear or branched (C₁-C₁₀) alkyl, or linear or branched (C₂-C₃₀) alkenyl and x is a number from 1 to 10,
—(CR⁴R⁵), naphthyl, wherein R⁴ and R⁵ are the same or different, and are —H, an OH group, linear or branched (C₁-C₁₀) alkyl or linear or branched (C₂-C₃₀) alkenyl and x is a number from 1 to 10,
a radical group of the formula (II) 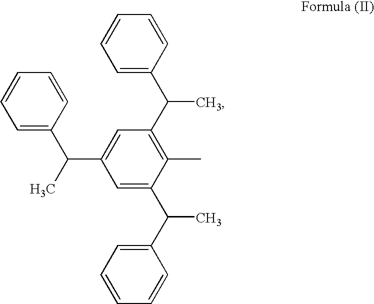
R⁶R⁷N—(CH₂)_{y}—, wherein R⁶ and R⁷ are the same or different, and are —H, linear or branched (C₁-C₁₀) alkyl, or linear or branched (C₂-C₃₀) alkenyl and y is a number from 1 to 22,
HO—(CH₂)_{y}—, wherein y is a number from 1 to 22,
—SO₃⁻X⁺, —PO₃²⁻X⁺X′⁺ or —CH₂COO⁻X⁺, wherein X⁺ and X′⁺ are H⁺, Li⁺, Na⁺, K⁺, Ca²⁺/2, Mg²⁺/2 or N(R⁸)₄⁺, and the R⁸ is the same or different and is —H or (C₁-C₁₀) alkyl,
a radical group of the formula (III) 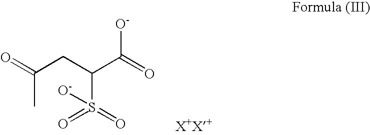
wherein X⁺ and X′⁺ are H⁺, Li⁺, Na⁺, K⁺, Ca²⁺/2, Mg²⁺/2 or N(R⁸)₄⁺, and the R⁸ is the same or different and is —H or (C₁-C₁₀) alkyl,
—C(R⁹)₂—COO⁻X⁺, —CO—R¹⁰—COO⁻X⁺ or —C(R⁹)₂C(R⁹)₂C(R⁹)₂—N(R¹¹)₂, wherein the R⁹ is the same or different and is —H and/or —CH₃, R¹⁰ is (C₁-C₁₀) alkylene or (C₂-C₃₀) alkenylene, the R¹¹ is the same or different and is (C₁-C₁₀) alkyl or (C₂-C₃₀) alkenyl, X⁺ is H⁺, Li⁺, Na⁺, K⁺, Ca²⁺/2, Mg²⁺/2 or N(R⁸)₄⁺, and the R⁸ is the same or different and is —H or (C₁-C₁₀) alkyl,
—C(R¹²)₂C(R¹²)₂C(R¹²)₂—N((GO)_{z}H)₂, wherein the R¹² is the same or different and is —H and/or —CH₃, G is —C₂H₄—, —C₃H₆— or —C₄H₈— and z is a number from 1 to 22, and/or
a radical group of the formula (IV) 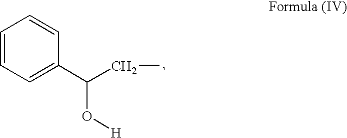
A, B and D, are the same or different, and are —C₂H₄—, —C₃H₆—, —C₄H₈— or —CH(phenyl)CH₂—,
the indices p1, q1, r1, p2, q2, r2, p3, q3 and r3 are numbers from 0 to 500,
and n is a number from 1 to 100,
with the proviso that at least one of the residues R¹, R² and R³ is a radical of the formula (II) or a radical of the formula (IV).

2. The aromatically substituted glycerine ether as claimed in claim 1, wherein at least one of the residues R¹, R² and R³ is a radical group of the formula (IV).

3. The aromatically substituted glycerine ether as claimed in claim 1, wherein n is a number from 1 to 35.

4. The aromatically substituted glycerine ether as claimed in claim 1, wherein A, B and D, are same or different, and are —C₂H₄— or —C₃H₆—.

5. The aromatically substituted glycerine ether as claimed in claim 1, wherein the indices p1, q1, r1, p2, q2, r2, p3, q3 and r3 are numbers from 0 to 100.

6. The aromatically substituted glycerine ether as claimed in claim 1, wherein the sum of the indices p1, q1, r1, p2, q2, r2, p3, q3 and r3 is a number from 0 to 100.

7. The aromatically substituted glycerine ether as claimed in claim 1, wherein the residues R¹, R² and R³ are —H or a radical of the formula (II) and at least one of the residues R¹, R² and R³ is a radical of the formula (II),
A, B and D, are the same or different, and are —C₂H₄—, —C₃H₆—, d—C₄H₈— or —CH(phenyl)CH₂—,
the indices p1, q1, r1, p2, q2, r2, p3, q3 and r3 are numbers from 0 to 100, and the sum of the indices p1, q1, r1, p2, q2, r2, p3, q3 and r3 is a number from 0 to 100,
n is a number from 1 to 8,
and when the residue R² is a radical of the formula (II), one or more of the residues R²is a radical of the formula (II).

8. The aromatically substituted glycerine ether as claimed in claim 1, wherein R¹ is a radical of the formula (II), R² and R³ are —H;
A, B and D, are same or different, and are —C₂H₄—, —C₃H₆—, —C₄H₈— or —CH(phenyl)CH₂—,
the indices p1, q1, r1, p2, q2, r2, p3, q3 and r3 are numbers from 0 to 100, the sum of the indices p1, q1, r1, p2, q2, r2, p3, q3 and r3 is a number from 0 to 100 and n is a number from 1 to 8.

9. The aromatically substituted glycerine ether as claimed in claim 1, wherein
at least one of the residues R¹, R² or R³ is a radical of the formula (III) and the other residues R¹, R² and R³ have the meaning stated in claim 1,
A, B and D, are the same or different, and are —C₂H₄—, —C₃H₆—, —C₄H₈— or —CH(phenyl)CH₂—,
the indices p1, q1, r1, p2, q2, r2, p3, q3 and r3 are numbers from 0 to 100, and the sum of the indices p1, q1, r1, p2, q2, r2, p3, q3 and r3 is a number from 0 to 100,
n is a number from 2 to 10,
and when the residue R² is not —H, just one or else also more of the residues R² may not be —H.

10. The aromatically substituted glycerine ether as claimed in claim 1, wherein R¹ is a radical of the formula (II), one of the residues R² or R³is a radical of the formula (III) and all remaining residues R² or R³ are —H,
A, B and D, are the same or different, and are —C₂H₄—, —C₃H₆—, —C₄H₈— or —CH(phenyl)CH₂—,
the indices p1, q1, r1, p2, q2, r2, p3, q3 and r3 are numbers from 0 to 100, and the sum of the indices p1, q1, r1, p2, q2, r2, p3, q3 and r3 is a number from 0 to 100, and n is for a number from 2 to 10

11. The aromatically substituted glycerine ether as claimed in claim 1, wherein
at least one of the residues R¹, R² and R³ is —PO₃²⁻X³⁰ X′⁺, wherein X⁺ and X′⁺ are H⁺, Li⁺, Na⁺, K⁺, Ca²⁺/2, Mg²⁺/2 or N(R⁸)₄⁺ and the R⁸ is the same or different and is —H or (C₁-C₁₀) alkyl, and the other residues R¹, R² and R³ have the meaning stated in claim 1,
A, B and D, are the same or different, and are —C₂H₄—, —C₃H₆—, —C₄H₈— or—CH(phenyl)CH₂—,
the indices p1, q1, r1, p2, q2, r2, p3, q3 and r3 are numbers from 0 to 100, and the sum of the indices p1, q1, r1, p2, q2, r2, p3, q3 and r3 is a number from 0 to 100;
n is a number from 2 to 10,
and when the residue R² is not —H, just one or else also more of the residues R² may not be —H.

12. The aromatically substituted glycerine ether as claimed in claim 1, wherein R¹ is a radical of the formula (II), one of the residues R² or R³ is —PO₃²⁻X⁺X′⁺, wherein X⁺ and X′⁺ are H⁺, Li⁺, Na⁺, K⁺, Ca²⁺/2, Mg²⁺/2 or N(R⁸)₄⁺ and the R⁸ is the same or different and is —H or (C₁-C₁₀) alkyl, and all remaining residues R² or R³ H,
A, B and D, are the same or different, and are —C₂H₄—, —C₃H₆—, —C₄H₈— or —CH(phenyl)CH₂—,
the indices p1, q1, r1, p2, q2, r2, p3, q3 and r3 are numbers from 0 to 100, and the sum of the indices p1, q1, r1, p2, q2, r2, p3, q3 and r3 is a number from 0 to 100, and n is a number from 2 to 10

13. The aromatically substituted glycerine ether as claimed in claim 1, wherein
at least one of the residues R¹, R² and R³ is —SO₃⁻X⁺, wherein X⁺ is H⁺, Li⁺, Na⁺, K⁺, Ca²⁺/2, Mg²⁺/2 or N(R⁸)₄⁺ and the R⁸ is the same or different and is —H or (C₁-C₁₀) alkyl, and the other residues R¹, R² and R³ have the meaning stated in claim 1,
A, B and D, are the same or different, and are —C₂H₄—, —C₃H₆—, —C₄H₈— or —CH(phenyl)CH₂—,
the indices p1, q1, r1, p2, q2, r2, p3, q3 and r3 are numbers from 0 to 100, and the sum of the indices p1, q1, r1, p2, q2, r2, p3, q3 and r3 is a number from 0 to 100,
n is a number from 2 to 10,
and when the residue R² is not —H, just one or else also more of the residues R² may not be —H.

14. The aromatically substituted glycerine ether as claimed in claim 1, wherein at least one of the residues R¹, R² and R³ is a radical of the formula (II) and at least one of the residues R¹, R² and R³ is —SO₃⁻X⁺, wherein X⁺ is H⁺, Li⁺, Na⁺, K⁺, Ca²⁺/2, Mg²⁺/2 or N(R⁸)₄⁺ and the R⁸ is the same or different and is —H or (C₁-C₁₀) alkyl, and all remaining residues R¹, R² or R³ are —H, A, B and D, are the same or different, and are —C₂H₄—, —C₃H₆—, —C₄H₈— or —CH(phenyl)CH₂—,
the indices p1, q1, r1, p2, q2, r2, p3, q3 and r3 are numbers from 0 to 100, and the sum of the indices p1, q1, r1, p2, q2, r2, p3, q3 and r3 is a number from 0 to 100, and n is a number from 2 to 10.

15. The aromatically substituted glycerine ether as claimed in o claim 1, wherein
the residues R¹, R² and R³ are —H and/or a radical of the formula (IV) and at least one of the residues R¹, R² and R³ is a radical of the formula (IV),
A, B and D is —CH₂CH₂—,
the indices p1, q1, r1, p2, q2, r2, p3, q3 and r3 are numbers from 0 to 25, and the sum of the indices p1, q1, r1, p2, q2, r2, p3, q3 and r3 is a number from 0 to 30,
n is a number from 1 to 5,
and wherein the radicals of the formula (IV) are bound directly to the glycerine unit and when the residue R² is a radical of the formula (IV), one or more of the residues R² is a radical of the formula (IV).

16. The aromatically substituted glycerine ether as claimed in claim 1, wherein R¹ and R² are a radical of the formula (IV), R³ is —H,
n is a number from 1 to 5,
p1, q1, r1, p2, q2, r2 are 0,
A, B and D are —CH₂CH₂—,
p3, q3 and r3 are numbers from 0 to 25, and the sum of the indices p3, q3 and r3 is a number from 0 to 30.

17. The aromatically substituted glycerine ether as claimed in claim 1, wherein
the residues R¹, R² and R³ are —H and/or a radical of the formula (IV) and at least one of the residues R¹, R² and R³ is a radical of the formula (IV),
A, B and D are —CH₂CH₂— and/or —CH(phenyl)CH₂—
and when the glycerine ethers contain both —CH₂CH₂— and —CH(phenyl)CH₂ radicals, —CH₂CH₂— is bound directly to the glycerine unit and —CH(phenyl)CH₂— to ethyleneoxy,
the indices p1, q1, r1, p2, q2, r2, p3, q3 and r3 are numbers from 0 to 25, and the sum of the indices p1, q1, r1, p2, q2, r2, p3, q3 and r3 is a number from 0 to 35, and
n stands for is a number from 1 to 5,
and wherein the radicals of the formula (IV) are bound directly to the glycerine unit and in the case where the residue R² is a radical of the formula (IV), one or more of the residues R² is a radical of the formula (IV).

18. The aromatically substituted glycerine ether as claimed in claim 1, wherein R¹ and R² are a radical of the formula (IV), R³ is —H,
n is a number from 1 to 5,
p1, q1 and r1 are 0,
A is —CH₂CH₂—,
B and D are —CH(phenyl)CH₂—,
p2, q2, r2, p3, q3 and r3 are numbers from 0 to 25, and the sum of the indices p2, q2, r2, p3, q3 and r3 is a number from 0 to 35.

19. A dispersion containing at least one aromatically substituted glycerine ether according to claim 1.

20. A surfactant comprising at least one aromatically substituted glycerine ether as claimed in claim 1.

21. (canceled)
